(19) United States
(12) Patent Application Publication
(10) Pub. No.: US 2010/0311159 A1
(43) Pub. Date: Dec. 9, 2010

(54) METHODS FOR PURIFYING OR DEPLETING MOLECULES OR CELLS OF INTEREST

(75) Inventor: Jacob Falewich, Ramat-HaSharon (IL)

Correspondence Address: MARTIN D. MOYNIHAN d/b/a PRTSI, INC., P.O. BOX 16446, ARLINGTON, VA, 22215, US

(73) Assignee: AFFISINK BIOTECHNOLOGY LTD., Kiryat-Ono (IL)

(21) Appl. No.: 12/808,679

(22) PCT Filed: Dec. 17, 2008
(86) PCT No.: PCT/IL08/01631

§ 371 Date: Jun. 17, 2010

Related U.S. Application Data
(60) Provisional application No. 61/006,067, filed on Dec. 17, 2007.

(51) Int. Cl. C12N 5/00; C12N 1/00; C07K 16/00

(52) U.S. Cl. 435/325; 435/243; 530/387.1

(57) ABSTRACT

A method of separating a target molecule or cell of interest from a sample is provided. The method comprising:

(a) contacting a sample including the target molecule or cell of interest with:

(i) a non-immobilized coordinator ion or molecule; and

(ii) a non-immobilized composition which comprises at least one ligand capable of binding directly or indirectly the target molecule or cell of interest, the at least one ligand being attached to at least two coordinating moieties selected capable of directing formation of a non-covalent complex when co-incubated with the non-immobilized coordinator ion or molecule and the target molecule or cell of interest,

wherein the contacting is effected in a solution having a predetermined volume; and

(b) applying a gravitational or centrifugal force on the solution, in a magnitude and a time period sufficient to concentrate at least 70% of the non-covalent complex in no more than 10% of the volume as a suspension, resulting in a solute phase separation between the no more than 10% of the volume and a remaining of the volume.

(c) collecting or disposing the no more than 10% of the volume, thereby separating the target molecule or cell of interest from the sample.

### FIELD AND BACKGROUND OF THE INVENTION

The present invention, in some embodiments thereof, relates to separation methods and use of same for purifying or depleting molecules or cells of interest.

Proteins and other macromolecules are increasingly used in research, diagnostics and therapeutics. Proteins are typically produced by recombinant techniques on a large scale with purification constituting the major cost (up to 60% of the total cost) of the production processes. Thus, large-scale use of recombinant protein products is hindered because of the high cost associated with purification.

Current protein purification methods are dependent on the use of a combination of various chromatography techniques. These techniques separate mixtures of proteins on the basis of their charge, degree of hydrophobicity or size among other characteristics. Several different chromatography resins are available for use with each of these techniques, allowing accurate tailoring of the purification scheme to the particular protein targeted for isolation. The essence of each of these separation methods is that proteins can either move at different rates down a long column, achieving a physical separation that increases as they pass further down the column, or selectively adhere to the separation medium, enabling differential elution by different solvents. In some cases, the column is designed such that contaminants bind thereto while the desired protein is found in the “flow-through.”

Affinity precipitation (AP) is the most effective and advanced approach for protein precipitation [Mattiasson (1998); Hilbrig and Freitag (2003) J Chromatogr B Analyt Technol Biomed Life Sci. 790(1-2):79-90]. Current state of the art AP employs ligand coupled “smart polymers”. “Smart polymers” [or stimuli-responsive “intelligent” polymers or Affinity Macro Ligands (AML)] are polymers that respond with large property changes to small physical or chemical stimuli, such as changes in pH, temperature, radiation and the like. These polymers can take many forms; they may be dissolved in an aqueous solution, adsorbed or grafted on aqueous-solid interfaces, or cross-linked to form hydrogels [Hoffman J Controlled Release (1987) 6:297-305; Hoffman Intelligent polymers. In: Park K, ed. Controlled drug delivery. Washington: ACS Publications, (1997) 485-98; Hoffman Intelligent polymers in medicine and biotechnology. Artif Organs (1995) 19:458-467]. Typically, when the polymer's critical response is stimulated, the smart polymer in solution will show a sudden onset of turbidity as it phase-separates; the surface-adsorbed or grafted smart polymer will collapse, converting the interface from hydrophilic to hydrophobic; and the smart polymer (cross-linked in the form of a hydrogel) will exhibit a sharp collapse and release much of its swelling solution. These phenomena are reversed when the stimulus is reversed, although the rate of reversion often is slower when the polymer has to redissolve or the gel has to re-swell in aqueous medium.

“Smart” polymers may be physically mixed with, or chemically conjugated to, biomolecules to yield a large family of polymer-biomolecule systems that can respond to biological as well as to physical and chemical stimuli. Biomolecules that may be polymer-conjugated include proteins and oligopeptides, sugars and polysaccharides, single- and double-stranded oligonucleotides and DNA plasmids, simple lipids and phospholipids, and a wide spectrum of recognition ligands and synthetic drug molecules.

A number of structural parameters control the ability of smart polymers to specifically precipitate proteins of interest; smart polymers should contain reactive groups for ligand coupling; not interact strongly with the contaminants; make the ligand available for interaction with the target protein; give complete phase separation of the polymer upon a change of medium property; form compact precipitates; exclude trapping of contaminants into the gel structure and be easily solubilized after the precipitate is formed.

Although many different natural as well as synthetic polymers have been utilized in AP [Mattiasson (1998) J. Mol. Recognit. 11:211] the ideal smart polymers remain elusive, as affinity precipitations performed with currently available smart polymers, fail to meet one or several of the above-described requirements [Hilbrig and Freitag (2003), supra].

The availability of efficient and simple protein purification techniques may also be useful in protein crystallization, in which protein purity extensively affects crystal growth. The conformational structure of proteins is a key to understanding their biological functions and to ultimately designing new drug therapies. The conformational structures of proteins are conventionally determined by x-ray diffraction from their crystals. Unfortunately, growing protein crystals of sufficient high quality is very difficult in most cases, and such difficulty is the main limiting factor in the scientific determination and identification of the structures of protein samples.

PCT Application WO2005/010141, PCT Application WO2006/085321 and U.S. Patent publication No. 2008-0108053 teach compositions and methods for purifying molecules, cells and viruses of interest. Basically, non-immobilized compositions are used for generating a non-covalent matrix which comprises the target molecule. The target molecule is associated with the matrix based on affinity recognition and the matrix is formed only following binding to the target molecule.

### SUMMARY OF THE INVENTION

According to an aspect of some embodiments of the present invention there is provided a method of purifying a target molecule or cell of interest, the method comprising:

(a) contacting a sample including the target molecule or cell of interest with:

(i) a non-immobilized coordinator ion or molecule; and

(ii) a non-immobilized composition which comprises at least one ligand capable of binding directly or indirectly the target molecule or cell of interest, the at least one ligand being attached to at least two coordinating moieties selected capable of directing formation of a non-covalent complex when co-incubated with the non-immobilized coordinator ion or molecule and the target molecule or cell of interest,

wherein the contacting is effected in a solution having a predetermined volume; and

(b) applying a gravitational or centrifugal force on the solution, in a magnitude and a time period sufficient to concentrate at least 70% of the non-covalent complex in no more than 10% of the volume as a suspension, resulting in a solute phase separation between the no more than 10% of the volume and a remaining of the volume.

(c) collecting the no more than 10% of the volume, thereby purifying the target molecule or cell of interest.

According to an aspect of some embodiments of the present invention there is provided a method of depleting a target molecule or cell of interest, the method comprising:

(a) contacting a sample including the target molecule or cell of interest with:

(i) a non-immobilized coordinator ion or molecule; and

(ii) a non-immobilized composition which comprises at least one ligand capable of binding directly or indirectly the target molecule or cell of interest, the at least one ligand being attached to at least two coordinating moieties selected capable of directing formation of a non-covalent complex when co-incubated with the non-immobilized coordinator ion or molecule and the target molecule or cell of interest,

wherein the contacting is effected in a solution having a predetermined volume; and

(b) applying a gravitational or centrifugal force on the solution, in a magnitude and a time period sufficient to concentrate at least 70% of the non-covalent complex in no more than 10% of the volume as a suspension, resulting in a solute phase separation between the no more than 10% of the volume and a remaining of the volume.

(c) removing the no more than 10% of the volume, thereby depleting the target molecule or cell of interest.

According to some embodiments of the invention, the molecule of interest is selected from the group consisting of a protein, a nucleic acid sequence, a small molecule chemical and an ion.

According to some embodiments of the invention, the target cell of interest is selected from the group consisting of a eukaryotic cell and a prokaryotic cell.

According to some embodiments of the invention, the at least one ligand is selected from the group consisting of a protein, a glycoprotein, a growth factor, a hormone, a nucleic acid sequence, an antibody, an epitope tag, an avidin, a biotin, a enzymatic substrate and an enzyme.

According to some embodiments of the invention, the coordinating moiety is selected from the group consisting of a chelator, a biotin, a nucleic acid sequence, an epitope tag, an electron poor molecule and an electron-rich molecule.

According to some embodiments of the invention, the non-immobilized coordinator ion or molecule is selected from the group consisting of a metal ion, an avidin, a nucleic acid sequence, an electron poor molecule and an electron-rich molecule.

According to some embodiments of the invention, the method further comprising recovering the target molecule or cell of interest from the no more than 10% of the volume.

According to some embodiments of the invention, the at least one ligand is a composite ligand which comprises a scaffold moiety attached to at least one target recognition moiety capable of directly or indirectly binding the target molecule or cell.

According to some embodiments of the invention, the scaffold moiety comprise albumin.

According to some embodiments of the invention, the albumin is selected from the group consisting of bovine serum albumin, Human serum albumin (HSA) and ovalbumin.

According to some embodiments of the invention, the target recognition moiety is selected from the group consisting of glutathione, a nucleic acid sequence, an amino acid sequence, a hormone, a histidine, a protease substrate, a protease inhibitor, a lectin, a LacI, a Cibarcon blue, a zinc finger protein and a chelator.

According to some embodiments of the invention, the at least one ligand is a composite ligand which comprises a scaffold moiety attached to at least one chelator molecule capable of indirectly binding the His-Tagged molecule via a metal ion.

According to some embodiments of the invention, the metal ion is different from the coordinator ion.

According to some embodiments of the invention, the contacting with (ii) is effected prior to (i).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of affinity purification according to some embodiments of the present invention. Step 1—A soluble non-immobilized ligand binds to the target and forms a soluble non-immobilized composition of matter (ii). Please note, that, the ligand is covalently bound to at least two coordinating moieties (A). Step 2: The soluble composition of matter (ii) becomes insoluble in the presence of an appropriate soluble non-immobilized coordinator ion or molecule (i). The non-covalent matrix thus formed can be separated from the original mixture under mild g force conditions.

FIGS. 2A-B are schematic illustrations of a spiral pipe outlets and traps which can be used according to some embodiments of the present invention;

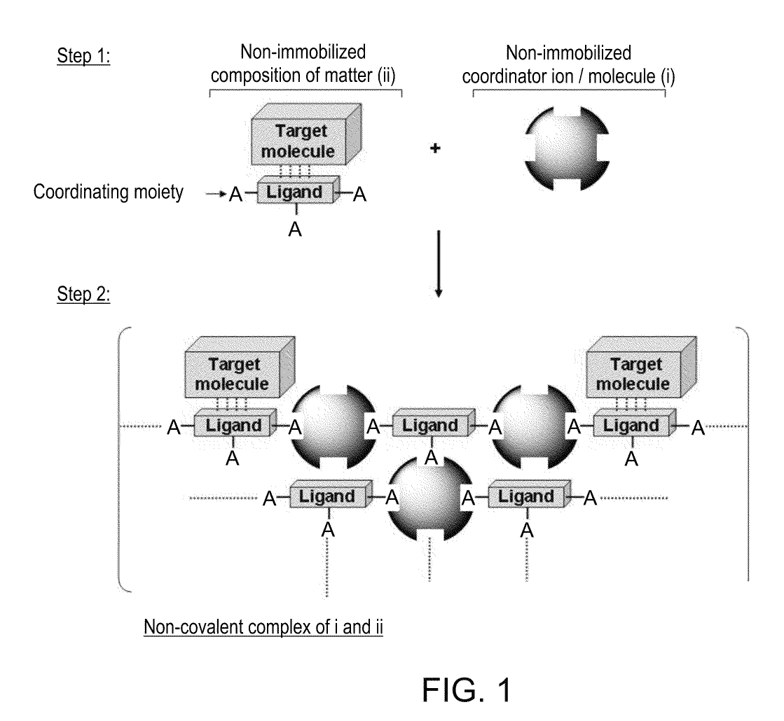

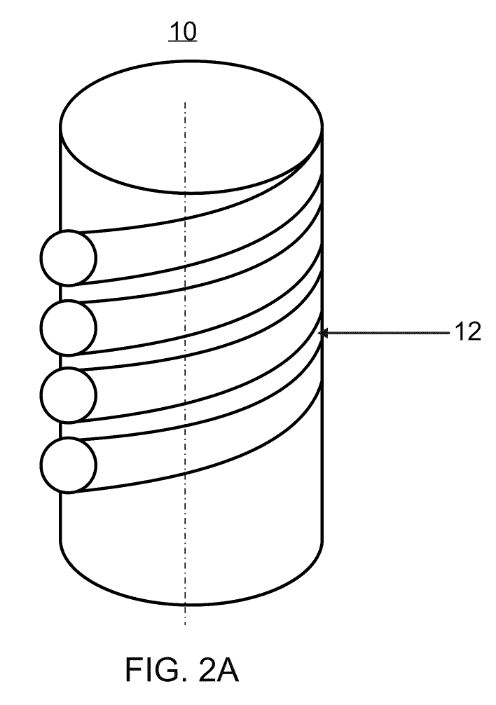

### DESCRIPTION OF EMBODIMENTS OF THE INVENTION

The present invention, in some embodiments thereof, relates to methods of purifying or depleting molecules or cells of interest.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The state of the art approach in protein purification is Affinity Chromatography, which is based on the use of solid phase or polymers coupled to an immobilized recognition unit, which binds the protein of interest resulting in binding of same on the solid phase. However, at present, the promise these heterogeneous systems has not been realized due to several drawbacks mainly, entrapment of impurities during the precipitation process and adsorption of impurities to the polymeric matrix.

PCT Application WO2005/010141, PCT Application WO2006/085321 and U.S. Patent publication No. 2008-0108053 teach non-immobilized compositions for generating a non-covalent matrix which comprises the target molecule. The matrix is formed only following binding to the target molecule. Matrices thus formed are of reduced level of contaminations, do not require the use of sophisticated laboratory equipment (FPLC) requiring high maintenance and do not lead to column fouling.

In an endeavor to improve the aforementioned process, the present inventors have uncovered, through laborious experimentation and screening a novel approach for isolating the matrix which allows simple recovery of the target molecule or cell therefrom without forming a precipitate. In doing so, isolation of the target molecule or cell is rendered faster in a batch process, easier to implement, reproducible and amenable to scaling up.

Thus, according to an aspect of the present invention there is provided a method of purifying a target molecule or cell of interest, the method comprising:

(a) contacting a sample including the target molecule or cell of interest with:

(i) a non-immobilized coordinator ion or molecule; and

(ii) a non-immobilized composition which comprises at least one ligand capable of binding directly or indirectly the target molecule or cell of interest, the at least one ligand being attached to at least two coordinating moieties selected capable of directing formation of a non-covalent complex when co-incubated with the non-immobilized coordinator ion or molecule and the target molecule or cell of interest;

wherein the contacting is effected in a solution having a predetermined volume; and

(b) applying a gravitational or centrifugal force on the solution, in a magnitude and a time period sufficient to concentrate at least 70% (e.g., 30%, 40%, 50%, 60%, 70%, 80%, 90% or even 100% or 30-100%, 50-100%, 50-90%, 70-90%, 70-80%) of the non-covalent complex in no more than 10% but no less than 0.01% of the volume, referred to herein as the “concentrated phase”, (e.g., no less than 0.1%, 0.5%, 3%, 4%, 5%, 7%, 8%, 9%) as a suspension, resulting in a solute phase separation between the no more than 10% of the volume and a remaining of the volume.

(c) collecting the concentrated phase, thereby purifying the target molecule or cell of interest.

As used herein the term “purifying” refers to at least separating the molecule or cell of interest from the sample (e.g., at least 30%, 40%, 50%, 60%, 70%, 80%, 90%, 92%, 94%, 96%, 98%, or even 100% separation) by changing its solubility upon generation of the non-covalent process (i.e., phase separation).

As used herein the term “sample” refers to a solution including the molecule, cell or virus of interest and possibly one or more contaminants (e.g., substances that are different from the desired molecule of interest, also referred to herein as impurities). For example when the molecule of interest is a secreted recombinant polypeptide, the sample can be the conditioned medium, which may include in addition to the recombinant polypeptide, serum proteins as well as metabolites and other polypeptides, which are secreted from the cells. When the sample includes no contaminants, purifying refers to concentrating.

The target molecule can be a macromolecule such as a protein (e.g., a prion), a carbohydrate, a glycoprotein, a lipid or a nucleic acid sequence (e.g. DNA such as plasmids, RNA) or a small molecule such as a chemical, a virus or a combination of same (e.g., toxins such as endotoxins or a chromatin). Although most of the examples provided herein describe proteinacious target molecules, it will be appreciated that the present invention is not limited to such targets.

The target cell can be a eukaryotic cell or a prokaryotic cell.

As mentioned, the ligand is capable of binding directly or indirectly the molecule or cell of interest.

As used herein the term “ligand” refers to a synthetic or a naturally occurring molecule preferably exhibiting high affinity (e.g. K_{D}<10⁻⁵) binding to the target molecule of interest and as such the two are capable of specifically interacting. In a direct configuration, the ligand binds the molecule/cell of interest directly. Thus, when the target of interest is a cell, the ligand is selected capable of binding a protein, a carbohydrate or chemical, which is present on the surface of the cell (e.g. cellular marker). Alternatively, the target molecule or cell may be labeled (e.g., with an antibody) and the ligand bind that label. The latter configuration is further described below. In an exemplary embodiment, ligand binding to the molecule or cell of interest is a non-covalent binding. The ligand according to this aspect of the present invention may be mono, bi (antibody, growth factor) or multi-valent ligand and may exhibit affinity to one or more molecules or cells of interest (e.g. bi-specific antibodies). In addition multiple ligands may be employed to purify different targets at the same purification process, for example to purify a number of growth factors from a sample, a mixture of antibodies with different specificities may be employed as the ligand. Examples of ligands which may be used in accordance with the present invention include, but are not limited to, antibodies, mimetics (e.g. Affibodies® see: U.S. Pat. Nos. 5,831,012, 6,534,628 and 6,740,734) or fragments thereof, epitope tags, antigens, biotin and derivatives thereof, avidin and derivatives thereof, metal ions, receptors and fragments thereof (e.g. EGF binding domain), enzymes (e.g. proteases) and mutants thereof (e.g. catalytic inactive), substrates (e.g. heparin), lectins (e.g. concanavalin A), carbohydrates (e.g. heparin), nucleic acid sequences [e.g. aptamers and Spiegelmers [Wlotzka® (2002) Proc. Natl. Acad. Sci. USA 99:8898-02], dyes which often interact with the catalytic site of an enzyme mimicking the structure of a natural substrate or co-factor and consisting of a chromophore (e.g. azo dyes, anthraquinone, or phathalocyanine), linked to a reactive group (e.g. a mono- or dichlorotriazine ring, see, Denzili (2001) J Biochem Biophys Methods. 49 (1-3):391-416), small molecule chemicals, receptor ligands (e.g. growth factors and hormones), mimetics having the same binding function but distinct chemical structure, or fragments thereof (e.g. EGF domain), ion ligands (e.g. calmodulin), protein A, protein G and protein L or mimetics thereof (e.g. PAM, see Fassina (1996) J. Mol. Recognit. 9:564-9], chemicals (e.g. cibacron Blue which bind enzymes and serum albumin; amino acids e.g. lysine and arginine which bind serine proteases) and magnetic molecules such as high spin organic molecules and polymers (see www.dotchemdotunldotedu/rajca/highspindothtml).

According to an exemplary embodiment the ligand is an antibody binding moiety. Such an antibody binding moiety can be any molecule which is capable of binding an immunoglobulin region of an antibody. Examples include but are not limited to protein A/G/L (or mimetics of same, e.g., MAbsorbent^{®}a Protein A mimetic—ProMetic Life Sciences Inc. (Canada), www.dotprometicdotcom/en/protein-technologies/bioseparation/mabsorbentsdotphp), as well as antibodies (e.g., secondary antibodies) or antibody fragments. Methods of generating antibodies or fragments of same are well known in the art.

According to another exemplary embodiment the ligand is a composite ligand composed of a scaffold/platform moiety attached to a target recognition moiety.

The scaffold/platform portion is typically an inert molecule which comprises sufficient active groups (e.g., amines) for conjugating the target recognition moieties.

The composite ligand is typically synthetic and the chemistry of synthesis depends on the active groups as well as on the nature of the target recognition moiety. Methods of synthesizing such composite ligands are well known in the art.

The target recognition moiety can be any affinity binding molecule of an affinity binding pair. The target recognition moiety may bind the target directly or indirectly.

The composite ligand approach is effected to provide a ligand with enhanced avidity by attaching target recognition moieties to a molecular scaffold/platform. Thus, the ligand is a composite (synthetic or natural) entity comprising a basically inert soluble scaffold/platform having active groups (e.g., amines) for chemically attaching the target recognition moieties as well as the target recognition moieties attached thereto. In accordance with an exemplary embodiment of the present invention the scaffold is albumin and the like e.g., BSA, HSA, ovalbumin. The target recognition moieties can be homogeneous (i.e., the same) or heterogeneous (i.e., not the same) exhibiting high affinity (e.g. K_{D}<10⁻⁵) binding to the target molecule of interest and as such the two are capable of specifically interacting. Binding of the target can be directly or indirectly (e.g., mediated by a metal). The composite ligand of the present invention is chemically bound to coordinating moieties.

The following provides exemplary application embodiments which can be used in accordance with the composite ligand teachings of the present invention.

a. GST-proteins with a: [Desthiobiotin-Albumin-Glutathione] conjugate (FIG. 52A).

b. Poly(A⁺) mRNA with a: [Desthiobiotin-Albumin-oligo(dT)] conjugate (FIG. 52B).

c. Membrane proteins (e.g., Na,K-ATPase) with a: [Desthiobiotin-Albumin-Ouabain] conjugate (FIG. 52C).

d. Depletion of pyrogens with a: [Desthiobiotin-Albumin-Histidine] conjugate (FIG. 52D).

e. Purification of ribonucleosides with a [Desthiobiotin-Albumin-Boronic acid] conjugate.

f. Isolation of C-reactive protein binding with a [Desthiobiotin-Albumin-p-Aminophenyl phosphoryl choline] conjugate.

g. Isolation of cathepsin D, rennin, pepsin, bacterial aspartic proteinases and HIV proteases with a [Desthiobiotin-Albumin-Pepstatin] conjugate.

h. Purification of nanoparticulates (e.g., protein inclusion bodies as enhanced-expression vehicles, Virus like particles as putative vaccine cores) or plasmid DNA. Plasmid DNA can be isolated with the following general conjugate:

[Desthiobiotin/Catechol: Albumin/or any other soluble protein or soluble entity capable of being modified: any moiety capable of interacting with plasmids].

Sequence specific interaction on an oligonucleotide capable of forming a triple helix with the plasmid:

### Desthiobiotin-Albumin-Sequence Specific Oligonucleotide

Binding to the plasmid via a zinc finger protein recognizing a specific nucleotide sequence which is either naturally present on is inserted to the plasmid.

### Desthiobiotin-Albumin-Zinc Finger Protein

Utilization of the LacI protein as a ligand:

### Desthiobiotin-Albumin-LacI

i. For Proteomic applications, simultaneous removal of high abundance proteins (e.g. Albumin, IgG's) from samples prior to their 2D gel electrophoresis analysis, utilizing a mixture of:

### Desthiobiotin-Albumin-Cibacron Blue+Desthiobiotinylated-Protein A Conjugates

According to an exemplary embodiment the composite ligand is capable of binding a His-tagged molecule, the at least one ligand being a composite ligand which comprises an scaffold moiety attached to at least one chelator molecule capable of indirectly binding the His-Tagged molecule via a metal ion, the at least one ligand being attached to at least two coordinating moieties selected capable of directing the composition-of-matter to form a non-covalent complex when co-incubated with a coordinator ion or molecule.

As used herein the phrase “coordinating moiety” refers to any molecule having sufficient affinity (e.g. K_{D}<10⁻⁵) to a coordinator ion or molecule. The coordinating moiety can direct the composition of matter of this aspect of the present invention to form a non-covalent complex when co-incubated with a coordinator ion or molecule. Examples of coordinating moieties which can be used in accordance with the present invention include but are not limited to, epitopes (antigenic determinants antigens to which the paratope of an antibody binds), antibodies, chelators (e.g. His-tag), biotin, nucleic acid sequences, protein A or G), electron poor molecules and electron rich molecules and other molecules described hereinabove (see examples for ligands).

It will be appreciated that a number of coordinating moieties can be bound to the ligand described above.

It will be further appreciated that different coordinating moieties can be attached to the ligand such as a chelator and an electron rich/poor molecule to form a complex. Such a combination of binding moieties may mediate the formation of polymers or ordered sheets (i.e., networks) containing the molecule of interest.

To avoid competition and/or further problems in the recovery of the molecule of interest from the complex, the coordinating moiety is selected so as to negate the possibility of coordinating moiety-ligand interaction or coordinating moiety-target molecule interaction. For example, if the ligand is an antigen having an affinity towards an immunoglobulin of interest then the coordinating moiety is preferably not an epitope tag or an antibody capable of binding the antigen.

As used herein the phrase “coordinator ion or molecule” refers to a soluble entity (i.e., molecule or ion), which exhibits sufficient affinity (i.e., K_{D}<10⁻⁵) to the coordinating moiety and as such is capable of directing the composition of matter of this aspect of the present invention to form a non-covalent complex. Examples of coordinator molecules which can be used in accordance with the present invention include but are not limited to, avidin and derivatives thereof, antibodies, electron rich molecules, electron poor molecules and the like. Examples of coordinator ions which can be used in accordance with the present invention include but are not limited to, mono, bis or tri valent metals. FIG. 25 illustrates examples of chelators and metals which can be used as a coordinator ion by the present invention. FIG. 26 lists examples of electron rich molecules and electron poor molecules which can be used by the present invention. Methods of generating antibodies and antibody fragments as well as single chain antibodies are described in Harlow and Lane, Antibodies: A Laboratory Manual, Cold Spring Harbor Laboratory, New York, 1988, incorporated herein by reference; Goldenberg, U.S. Pat. Nos. 4,036,945 and 4,331,647, and references contained therein; See also Porter, R. R. [Biochem. J. 73: 119-126 (1959); Whitlow and Filpula, Methods 2: 97-105 (1991); Bird et al., Science 242:423-426 (1988); Pack et al., Bio/Technology 11:1271-77 (1993); and U.S. Pat. No. 4,946,778].

According to embodiments of the present invention, the ligand, coordinating moiety and coordinator ion or molecule are provided soluble (i.e., non-immobilized).

The ligand of this aspect of the present invention may be bound directly to the coordinating moiety, depending on the chemistry of the two. Measures are taken, though, to maintain recognition (e.g. affinity) of the ligand to the molecule of interest. When needed (e.g. steric hindrance), the ligand may be bound to the coordinating moiety via a linker. A general synthetic pathway for modification of representative chelators with a general ligand is shown in FIG. 14. Margherita et al. (1993) J. Biochem. Biophys. Methods 38:17-28 provides synthetic procedures which may be used to attach the ligand to the coordinating moiety of the present invention.

When the ligand and coordinating moiety bound thereto are both proteins (e.g. growth factor and epitope tag, respectively), synthesis of a fusion protein can be effected by molecular biology methods (e.g. PCR) or biochemical methods (solid phase peptide synthesis).

Complexes of the present invention may be of various complexity levels, such as, monomers, dimers, polymers, sheets and lattices which may form three dimensional (3D) structures. It is well established that the higher complexity of the complex the more rigid is the structure enabling use thereof in crystallization procedures for example. Furthermore, large complexes will phase separate more rapidly, negating the use of further centrifugation steps.

According to exemplary embodiments of the present invention, the ligand is selected such that the target molecule/cell is uniformly bound thereto. For example, the ligand can be selected such that the target molecule/cell bound by the complex is only associated with a single ligand molecule of the complex or with a predetermined number of ligand molecules. As is further described below, such uniform association between ligand and target molecule/cell ensures that purification of the target from the complex is uniform, i.e. that a single elution step releases substantially all of the complex-bound target and allows working in batch configurations.

Examples of ligand configuration which enable such uniform binding of the target molecule/cell, include: peptides (i.e., cyclic or linear), Protein A or G or L, antibodies, lectines (e.g., concanavalin A from Jack bean, Jacalin from Jack fruit), various dyes (e.g., Cibacron Blue 3GA) and aptamers.

The sample may be pre-treated such that the molecule or cell of interest are labeled (e.g., such as with an antibody, whereby the ligand is an antibody binding moiety attached to at least 2 coordinating moieties).

In order to initiate purification, the ligand is contacted with the sample. This may be effected by adding the ligand attached to the coordinating moiety to the sample allowing binding of the molecule of interest directly or indirectly to the ligand and then adding the coordinator ion or molecule to allow complex formation. However, it will be appreciated that the ligand and coordinator ion or molecule may be simultaneously added to the sample. Further alternatively, the coordinator ion or molecule may be added first followed by addition of the ligand. When the ligand is added first, and in order to avoid rapid formation of complexes (which may result in the entrapment of contaminants) slow addition of the coordinator to the sample may be effected while stirring. Controllable rate of complex formation can also be achieved by adding free coordinating entity (i.e., not bound to the ligand), which may also lead to the formation of smaller complexes which may be beneficial in a variety of applications such as for the formation of immunogens.

As mentioned, once the complex is formed (seconds, minutes to hours), a gravitational or centrifugal force is applied on said solution, in a magnitude and a time period sufficient to concentrate at least 30% of said non-covalent complex in no more than 10% of said volume as a suspension (i.e., concentrated phase), resulting in a solute phase separation between said concentrated phase and the remaining of the volume.

The volume of the solution much depends on intended use and may vary in an exemplary embodiment from a few microliters to milliliters to liters. Importantly even after applying the above mentioned gravitational or centrifugal force, the concentrated phase maintains its suspension properties, essentially, no precipitate is formed.

As used herein the term “suspension” refers to a mixture in which fine particles are suspended in a fluid where they are supported by buoyancy

As used herein the term “precipitate” refers to an insoluble solid, alternatively the term precipitate refers to a substance suspended in less than 0.01% fluid.

Numerous methods are known in the art for phase separation. Examples include but are not limited to centrifugation at low g or the use of hydrocyclones.

The skilled artisan will know the g values for obtaining the above described concentrated phase (e.g., up to 2500×g for 1 minute).

In a specific embodiment, hydrocyclone technology can be used for phase separation. A hydrocyclone is a device to classify/separate or sort particles in a liquid suspension based on the densities of the particles. A hydrocyclone may be used to separate solids from liquids or to separate liquids of different density. A hydrocyclone will normally have a cylindrical section at the top where liquid is being fed tangentially, and a conical base. The angle, and hence length of the conical section, plays a role in determining operating characteristics.

A hydrocyclone has two exits on the axis: the smaller on the bottom (underflow or reject) and a larger at the top (overflow or accept). The underflow is generally the denser or thicker fraction, while the overflow is the lighter or more fluid fraction. Internally, centrifugal force is countered by the resistance of the liquid, with the effect that larger or denser particles are transported to the wall for eventual exit at the reject side with a limited amount of liquid, whilst the finer, or less dense particles, remain in the liquid and exit at the overflow side through a tube extending slightly into the body of the cyclone at the center. Forward hydrocyclones remove particles that are denser than the surrounding fluid, while reverse hydrocyclones remove particles that are less dense than the surrounding fluid. In a reverse hydrocyclone the overflow is at the apex and the underflow at the base. There are also parallel flow hydrocyclones where both the accept and reject are removed at the apex. Parallel-flow hydrocyclones remove particles that are lighter than the surround fluid. Hydrocyclones can be made of metal (mostly steel), ceramic or plastic (such as polyurethane, polypropylene, or other types). Metal or ceramic hydrocyclones are used for situations requiring more strength, or durability in terms of heat or pressure. In a suspension of particles with the same density, a relatively sharp cut can be made. The size at which the particles separate is a function of cyclone diameter, exit dimensions, feed pressure and the relative characteristics of the particles and the liquid. Efficiency of separation is a function of the solids' concentration: the higher the concentration, the lower the efficiency of separation. There is also a significant difference in suspension density between the base exit (fines) and the apex exit, where there is little liquid flow (see e.g., U.S. Pat. No. 5,071,556).

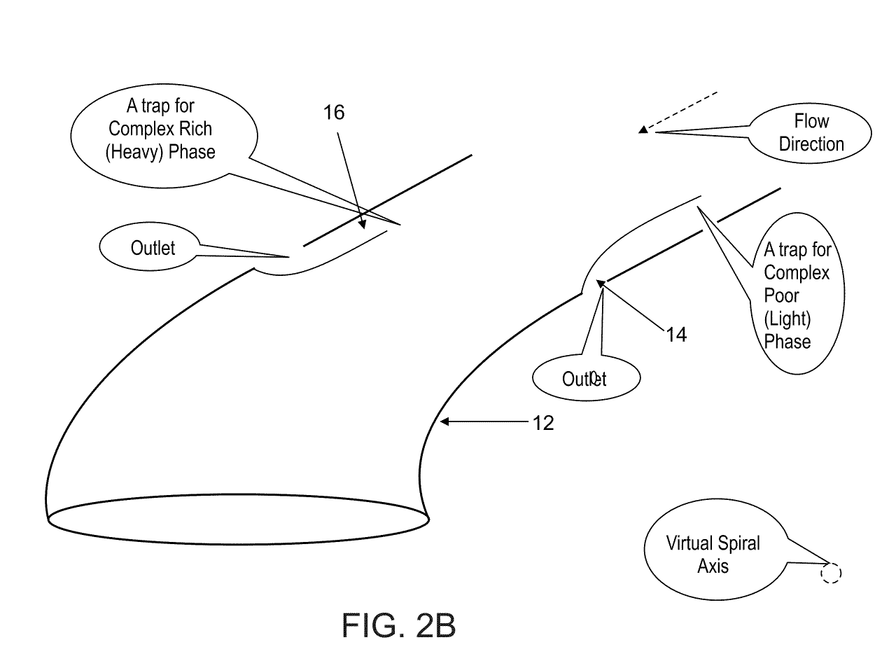

A specific configuration of phase separation means is shown in FIGS. 2a-b. Device 10 shows a spiral pipe structure. The complexes are forced by gravitational force within spiral 12 to the outside portion of the flow creating a complex rich (heavy) phase. A complex poor (light) phase is created within the inside portion of the flow (closer to the spiral axis). Small outlet openings 14 and traps 16 located down the pipe turns (at the pipe wall farther from the spiral axis) collect the complex rich phase of the sample, conveying it to a collection container or another pipe. Other outlet openings located down the pipe turns (at the pipe wall closer the spiral axis) collect the complex poor phase of the sample, conveying it to another collection container or yet another pipe. Inlets located along the pipe allow the addition of clean reaction solution (e.g., buffer) or other reagents. The collected complex rich solution may be further circulated via similar spiral pipes structures, to obtain a desired purity and concentration of the complexes. The collected complex poor solution may be further processed to recover more of the complexes.

Depending on the intended use of the molecule of interest, the concentrated volume may be subjected to further purification steps in order to recover the molecule of interest from the complex. This may be effected by using a number of biochemical methods which are well known in the art. Examples include, but are not limited to, fractionation on a hydrophobic interaction chromatography (e.g. on phenyl sepharose), ethanol precipitation, isoelectric focusing, reverse phase HPLC, chromatography on silica, chromatography on heparin sepharose, anion exchange chromatography, cation exchange chromatography, chromatofocusing, SDS-PAGE, ammonium sulfate precipitation, hydroxylapatite chromatography, gel electrophoresis, dialysis, and affinity chromatography (e.g. using protein A, protein G, an antibody, a specific substrate, ligand or antigen as the capture reagent).

It will be further appreciated that any of the above-described purification procedures may be repetitively applied on the sample (i.e., phase separation e.g., re-suspending the concentrated phase) to increase the yield and or purity of the target molecule.

Preferably, the composition of matter and coordinator ion or molecule are selected so as to enable rapid and easy isolation of the target molecule from the complex formed. For example, the molecule of interest may be eluted directly from the complex, provided that the elution conditions employed do not disturb binding of the coordinating moiety to the coordinator. For example, when the coordinating moiety used in the complex is a chelator, high ionic strength may be applied to elute the molecule of interest, since it is well established that it does not effect metal-chelator interactions. Alternatively, elution with chaotropic salt may be used, since it has been shown that metal-chelator interactions are resistant to high salt conditions enabling elution of the target molecule at such conditions [Porath (1983) Biochemistry 22:1621-1630]. In the elution step additional stages of centrifugation or filtration may be employed.

On top of their purifying capabilities, the present methodology may also be used to deplete a sample from undesired molecules or cells.

Thus according to an aspect of the present invention there is provided a method of depleting a target molecule or cell of interest, the method comprising:

(a) contacting a sample including the target molecule or cell of interest with:

(i) a non-immobilized coordinator ion or molecule; and

(ii) a non-immobilized composition which comprises at least one ligand capable of binding directly or indirectly the target molecule or cell of interest, the at least one ligand being attached to at least two coordinating moieties selected capable of directing formation of a non-covalent complex when co-incubated with the non-immobilized coordinator ion or molecule and the target molecule or cell of interest,

wherein the contacting is effected in a solution having a predetermined volume; and

(b) applying a gravitational or centrifugal force on the solution, in a magnitude and a time period sufficient to concentrate at least 70% (e.g., 30%, 40%, 50%, 60%, 70%, 80%, 90% or even 100% or 30-100%, 50-100%, 50-90%, 70-90%, 70-80%) of the non-covalent complex in no more than 10% but no less than 0.01% of the volume, referred to herein as the “concentrated phase”, (e.g., no less than 0.1%, 0.5%, 3%, 4%, 5%, 7%, 8%, 9%) as a suspension, resulting in a solute phase separation between the no more than 10% of the volume and a remaining of the volume.

(c) removing the no more than 10% of the volume (i.e., concentrated phase), thereby depleting the target molecule or cell of interest.

This method have various uses such as in depleting tumor cells from bone marrow samples, depleting B cells and monocytes for the isolation and enrichment of T cells and CD8⁺cells or CD 4⁺cells from peripheral blood, spleen, thymus, lymph or bone marrow samples, depleting pathogens and unwanted substances (e.g. prions, toxins) from biological samples, protein purification (e.g. depleting high molecular weight proteins such as BSA) and the like.

As mentioned hereinabove multiple ligands may be employed for the depletion of a number of targets from a given sample such as for the removal of highly abundant proteins from biological fluids (e.g. albumin, IgG, anti-trypsin, IgA, transferrin and haptoglobin, see wwwdotchemdotagilentdotcom/cag/prod/ca/51882709smalldotpdf).

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

### Protein Purification According to the Present Teachings

According to some embodiments of the present invention, antibody purification is effected by phase separation which avoids the formation of a precipitate. This procedure involves the following method steps when applied on IgG:

1. IgG sinking.

2. Phase separation.

3. IgG retrieval.

### Materials and Methods

### Materials

Desthio-biotin (DB)-4-Protein A—7 mg/ml; Avidin 10 mg/ml ; NaPi pH67 50 mM; NaPi pH 7 100 mM; Glycine pH 3 100 mM; TRIS 1.6 m (not buffered); Unless otherwise indicated all chemicals were obtained Sigma-Aldrich, Rehovot, Israel.

Serum—IgG containing serum was diluted with NaPi pH 7 50 mM to a final concentration 10 mg/ml IgG.

Experimental Procedure:

### Complex Formation

The above mentioned chemicals were pre-warmed to room temperature (RT). 125 μl rabbit serum were added to a 1.5 Eppendorf tube. To the tube, 24 μl desthiobiotinylated Protein A (7 mg/ml) and 260 μl NaPi 50 m were added in that order. The resultant reaction mixture was mixed by pipetting up and down for 5 times. The reaction mixture was incubated for 10 minutes at room temperature. Thereafter, 92 μl Avidin (10 mg/ml) were added and immediately mixed by pipetting up and down for 5 times (the solution became cloudy). The mixture was then incubated at RT for 3 minutes.

### Phase Separation

The Eppendorf tube was spun at 2,500×g for 1 minute resulting in a distinct clear supernatant phase which appeared on top of a lower cloudy phase. The upper phase was discarded. Thereafter, 200 μl of NaPi pH 7 100 mM were added and pipetted up and down 5 times. The Eppendorf tube was then spun at 2,500×g for 1 minutes followed by discarding carefully the upper phase.

### IgG Retrieval

600 μl of glycine pH3 100 mM were added to the isolated lower phase. The solution was mixed by pipetting up and down 5 times and making sue that all aggregates are fully dissolved. The tube was allowed to rest for 5 min at RT and thereafter subjected to spinning at 14,000×g for 1 min. 500 μl of the supernatant were transferred into a fresh tube containing non-buffered Tris 1.6 M. This tube contained purified ready to use IgG at a physiological pH. For Up—Scaling volumes of the aforementioned reagents are linearly increased.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of purifying a target molecule or cell of interest, the method comprising:
(a) contacting in a solution a sample including the target molecule or cell of interest with:
(i) at least one non-immobilized ligand covalently attached to at least two coordinating moieties; and
(ii) a soluble non-immobilized coordinator ion or molecule capable of non-covalently binding said coordinating moieties;
wherein said contacting is effected in a predetermined volume thereby forming a suspension comprising a complex which comprises said coordinator ion or molecule non-covalently bound to said coordinating moieties and said ligand bound to said target molecule; and
(b) applying a gravitational or centrifugal force on said suspension, in a magnitude and a time period sufficient to concentrate at least 70% of said complex in no more than 10% of said volume as a suspension, resulting in a solute phase separation between said no more than 10% of said volume comprising at least 70% of said complex and the remaining no less than 90% of said volume, and
(c) collecting said no more than 10% of said volume, thereby purifying the target molecule or cell of interest.

2. A method of depleting a target molecule or cell of interest, the method comprising:
(a) contacting in a solution a sample including the target molecule or cell of interest with:
(i) at least one non-immobilized ligand covalently attached to at least two coordinating molecules; and
(ii) a soluble non-immobilized coordinator ion or molecule non-covalently binding said coordinating moieties
wherein said contacting is effected in a predetermined volume thereby forming a suspension comprising a complex which comprises said coordinator ion or molecule non-covalently bound to said coordinating moieties and said ligand bound to said target molecule; and
(b) applying a gravitational or centrifugal force on said suspension, in a magnitude and a time period sufficient to concentrate at least 70% of said complex in no more than 10% of said volume as a suspension, resulting in a solute phase separation between said no more than 10% of said volume comprising 70% of said complex and the remaining no less than 90% of said volume.
(c) removing said no more than 10% of said volume, thereby depleting the target molecule or cell of interest.

3. The method of claim 1, wherein the molecule of interest is selected from the group consisting of a protein, a nucleic acid sequence, a small molecule chemical and an ion.

4. The method of claim 1, wherein the target cell of interest is selected from the group consisting of a eukaryotic cell and a prokaryotic cell.

5. The method of claim 1, wherein said at least one ligand is selected from the group consisting of a protein, a glycoprotein, a growth factor, a hormone, a nucleic acid sequence, an antibody, an epitope tag, an avidin, a biotin, a enzymatic substrate and an enzyme.

6. The method of claim 1, wherein said coordinating moiety is selected from the group consisting of a chelator, a biotin, a nucleic acid sequence, an epitope tag, an electron poor molecule and an electron-rich molecule.

7. The method of claim 1, wherein said non-immobilized coordinator ion or molecule is selected from the group consisting of a metal ion, an avidin, a nucleic acid sequence, an electron poor molecule and an electron-rich molecule.

8. The method of claim 1, further comprising recovering the target molecule or cell of interest from said no more than 10% of said volume.

9. The method of claim 1, wherein said at least one ligand is a composite ligand which comprises a scaffold moiety attached to at least one target recognition moiety capable of directly or indirectly binding the target molecule or cell.

10. The method of claim 9, wherein said scaffold moiety comprise albumin.

11. The method of claim 10, wherein said albumin is selected from the group consisting of bovine serum albumin, Human serum albumin (HSA) and ovalbumin.

12. The method of claim 9, wherein said target recognition moiety is selected from the group consisting of glutathione, a nucleic acid sequence, an amino acid sequence, a hormone, a histidine, a protease substrate, a protease inhibitor, a lectin, a LacI, a Cibacron blue, a zinc finger protein and a chelator.

13. The method of claim 1, wherein said at least one ligand is a composite ligand which comprises a scaffold moiety attached to at least one chelator molecule capable of indirectly binding the His-Tagged molecule via a metal ion.

14. The method of claim 13, wherein said metal ion is different from said coordinator ion.

15. The method of claim 1, wherein said contacting with (i) is effected prior to (ii).

16. The method of claim 1, wherein said gravitational or centrifugal force is 2,500×g and said period of time is 1 minute.

17. The method of claim 2, wherein the molecule of interest is selected from the group consisting of a protein, a nucleic acid sequence, a small molecule chemical and an ion.

18. The method of claim 2, wherein said at least one ligand is selected from the group consisting of a protein, a glycoprotein, a growth factor, a hormone, a nucleic acid sequence, an antibody, an epitope tag, an avidin, a biotin, a enzymatic substrate and an enzyme.

19. The method of claim 2, wherein said coordinating moiety is selected from the group consisting of a chelator, a biotin, a nucleic acid sequence, an epitope tag, an electron poor molecule and an electron-rich molecule.

20. The method of claim 2, wherein said non-immobilized coordinator ion or molecule is selected from the group consisting of a metal ion, an avidin, a nucleic acid sequence, an electron poor molecule and an electron-rich molecule.

21. The method of claim 2, further comprising recovering the target molecule or cell of interest from said no more than 10% of said volume.

22. The method of claim 2, wherein said at least one ligand is a composite ligand which comprises a scaffold moiety attached to at least one chelator molecule capable of indirectly binding the His-Tagged molecule via a metal ion.

23. The method of claim 22, wherein said metal ion is different from said coordinator ion.

24. The method of claim 2, wherein said contacting with (i) is effected prior to (ii).

25. The method of claim 2, wherein said gravitational or centrifugal force is 2,500×g and said period of time is 1 minute.
